(19) United States
(12) Patent Application Publication
(10) Pub. No.: US 2017/0319740 A1
(43) Pub. Date: Nov. 9, 2017

(54) Bioactive Wound Dressing and Teeth Coating Based on Morphogenetically Active Amorphous Calcium Polyphosphate

(71) Applicant: NanotecMARIN GmbH, Mainz (DE)

(72) Inventor: Werner Ernst Ludwig Georg MÜLLER, Mainz (DE)

(73) Assignee: NanotecMARIN GmbH, Mainz (DE)

(21) Appl. No.: 15/527,553

(22) PCT Filed: Nov. 12, 2015
(86) PCT No.: PCT/EP2015/076468

§ 371 Date: May 17, 2017

(30) Foreign Application Priority Data
Nov. 17, 2014 (GB) 1420363.2
Apr. 1, 2015 (GB) 1505629.4
Jun. 19, 2015 (GB) 1510772.5

(51) Int. Cl. A61L 27/12; A61K 8/67; A61K 9/00; A61K 6/00; A61K 9/50; A61K 9/70; A61K 9/14; A61Q 11/00; A61L 26/00

(52) U.S. Cl. CPC A61L 27/12; A61K 9/70; A61K 8/671; A61K 9/0024; A61K 9/14; A61K 6/0017; A61K 9/501; A61L 26/00; A61L 2400/12; A61L 2430/12; A61Q 11/00

(57) ABSTRACT

This invention relates to a method for sealing dentinal tubules exposed at the tooth surface as a consequence of enamel defects, based on amorphous calcium polyphosphate (Ca-polyP) nano- or microparticles that strongly bind both to tooth enamel, cementum and dentin surfaces. The inventive method can also be used for the production of morphogenetically active tooth implants. A further aspect of this invention concerns the incorporation of such nano- or microparticles, after encapsulation of retinol (“retinol/aCa-polyP nano- or microspheres”), into wound dressings and related materials that are made, e.g., by electrospinning. The resulting, inventive retinol/aCa-polyP nano- or microspheres fiber mats show antimicrobial and wound healing properties and was found to increase the expression of the genes encoding for leptin and the leptin receptor, as well as the fatty acid binding protein 4 (FABP4) in a synergistic manner. This inventive material is the first material that can be used to promote wound healing through affecting the leptin/leptin receptor expression.

This invention relates to a method for sealing dentinal tubules exposed at the tooth surface as a consequence of enamel defects, based on amorphous calcium polyphosphate (Ca-polyP) nano- or microparticles that strongly bind both to tooth enamel, cementum and dentin surfaces. The inventive method can also be used for the production of morphogenetically active tooth implants. A further aspect of this invention concerns the incorporation of such nano- or microparticles, after encapsulation of retinol (“retinol/aCa-polyP nano- or microspheres”), into wound dressings and related materials that are made, e.g., by electrospinning. The resulting, inventive retinol/aCa-polyP nano- or microspheres fiber mats show antimicrobial and wound healing properties and was found to increase the expression of the genes encoding for leptin and the leptin receptor, as well as the fatty acid binding protein 4 (FABP4) in a synergistic manner. This inventive material is the first material that can be used to promote wound healing through affecting the leptin/leptin receptor expression.

### BACKGROUND OF THE INVENTION

Dental caries and tooth hypersensitivity belong to the most common diseases worldwide. The costs for the public health system caused by them are immense. As result of the loss of enamel or cementum dentinal tubules become exposed to the tooth surface. The consequences are an increased risk of a series of dental diseases/impairments, such as dentin hypersensitivity, caries, and pulp inflammation. Demineralization of enamel and cementum are caused by bacterial bio film formation, especially by the acid-producing Streptococcus mutans.

Physiologically, teeth enamel and dentin undergo a permanent remodeling by demineralization and remineralization processes. However, these processes, in particular tooth repair are slow. Calcium and phosphate ions, as well as by fluoride can be administered in order to partially reconstitute the crystal remnants on the subsurface lesions remaining after demineralization. The remineralized crystals are more resistant to acid, but less soluble and more brittle than the original mineral. The biomineral of tooth enamel, dentin and cementum mainly consists of hydroxyapatite (HA). The formation of HA deposits is controlled, among others, by certain growth factors (e.g., amelogenin and ameloblastin) and enzymes (e.g., ALP and carbonic anhydrase).

Dentin is traversed by a network of tubular structures, termed dentinal tubules. These tubules are shielded by the enamel (crown) and the cementum (root), which form a protective layer of the pulp against external physical and chemical influences, like temperature changes and acids, and prevent affection of the nerve protrusions and dentin hypersensitivity. The diameter of the dentinal tubules which protrude into the dentin layer and are open to the dental surface varies between 1 and 2.5 μm. Patients suffering from tooth hypersensitivity have larger number of open dentinal tubules and/or tubules with a larger in diameter than normal.

Strategies that have been undertaken to reseal and to desensitize dentinal tubules comprise: Occlusion of the dentinal tubules with Na-oxalate (Greenhill J D, Pashley D H (1981) The effects of desensitizing agents on the hydraulic conductance of human dentin in vitro. J Dent Res 60:686-698; Rusin R P, et al. (2010) Effect of a new desensitizing material on human dentin permeability. Dent Mater 26:600-607). However: The minerals formed are slowly dissolved in artificial saliva (Suge T, et al. (1995) Duration of dentinal tubule occlusion formed by calcium phosphate precipitation method: In vitro evaluation using synthetic saliva. J Dent Res 74:1709-1714).

Application of Na-fluoride which undergoes transient formation of crystals that remain only relatively loosely attached on the surface (Schlueter N, et al. (2009) Tin-containing fluoride solutions as anti-erosive agents in enamel: An in vitro tin-uptake, tissue-loss, and scanning electron micrograph study. Eur J Oral Sci 117:427-434).

Occlusion of the more surface-exposed sections (up to 10 μm deep) of dentinal tubules by the resealing of the teeth surfaces with products present in, e.g. Sensodyne, NovaMin and Colgate Sensitive Pro-Relief (Petrou I, et al. (2009) A Breakthrough therapy for dentin hypersensitivity: How dental products containing 8% arginine and calcium carbonate work to deliver effective relief of sensitive teeth. J Clin Dent 20:23-31; Ayad F, et al. (2009) Comparing the efficacy in reducing dentin hypersensitivity of a new toothpaste containing 8.0% arginine, calcium carbonate, and 1450 ppm fluoride to a commercial sensitive toothpaste containing 2% potassium ion: An eight-week clinical study on Canadian adults. J Clin Dent 20:10-16; Vahid-Golpayegani M, et al. (2012) Remineralization effect of topical novamin versus sodium fluoride (1.1%) on caries-like lesions in permanent teeth. J Dent (Tehran) 9:68-75). However: These attempts have been found not to be sufficient as a protection against the daily mechanical forces towards which the teeth are exposed (Moritz A, et al. (1998) Long-term effects of Co₂ laser irradiation on treatment of hypersensitive dental necks: Results of an in vivo study. J Clin Laser Med Surg 16:211-215; Orchardson R, Gilliam D (2006) Managing dentin hypersensitivity. J Am Dent Assoc 137:990-998; Chiang Y C, et al. (2014) A mesoporous silica biomaterial for dental biomimetic crystallization. ACS Nano 8:12502-12513).

Also, the market for the other application of the inventive product, the wound healing market, is rapidly growing. In 2021, the world wound management market has been estimated to reach to over $18.5 billion. The main market segment is the market for advanced wound care and closure for ulcer treatment, in particular the market for the diabetic foot ulcer management. The process of wound healing comprises at least four phases (reviewed in: Guo S, Dipietro L A. Factors affecting wound healing. J Dent Res 2010; 89: 219-229):

a) Hemostasis, b) Inflammation, c) Proliferation, and d) Remodeling.

PHASE 1: During the hemostasis period that immediately starts after wound setting vascular constriction and fibrin clot formation occurs.

PHASE 2: After bleeding is under control, pro-inflammatory cytokines and growth factors, including platelet-derived growth factor, fibroblast growth factor and epidermal growth factor, are released. In parallel, a sequential infiltration of neutrophils, macrophages and lymphocytes, as well as platelets takes place that contributes to the prevention of blood loss. The function of these cells is the elimination of invading microorganisms as well as the removal of cellular debris within the damaged tissue region. These cells produce and release morphogenetically active polymers, e.g. polyphosphate (polyP) from platelets (Morrissey J H, Choi S H, Smith S A. Polyphosphate: an ancient molecule that links platelets, coagulation, and inflammation. Blood 2012; 119: 5972-5979; Faxalv L, et al. Putting polyphosphates to the test: evidence against platelet-induced activation of factor XII. Blood 2013; 122: 3818-3824). PHASE 3: During granulation tissue formation, especially during the early phase of cutaneous wound repair, new capillaries endowing the neostroma with its granular appearance and macrophages, fibroblasts, and blood vessels move into the wound.

PHASE 4: Remodeling occurs that results in scar formation. Especially, during this phase the interaction of the cells, involved in remodeling, requires, for functional regeneration, the synthesis of the extracellular matrix by fibroblasts.

Major factors that influence wound healing include:

(i) oxygenation/superoxide radical formation, involved in energy production and/or oxidative killing of pathogens, and/or

(ii) infections via microorganisms which are normally restricted to the skin surface but might invade the underlying tissue strata.

Cytokines and growth factors that are released during wound healing initiate and maintain the interaction between the cells and control the regenerative response of the infiltrating cells. Besides of these protein factors retinoic acid and its precursor retinol are efficient regeneration-promoting stimuli (Jetten A M. Multi-stage program of differentiation in human epidermal keratinocytes: Regulation by retinoids. J Invest Dermatol 1990; 95: 44S-46S; Fisher G J & Voorhees J J. Molecular mechanisms of retinoid actions in skin. FASEB J 1996; 10: 1002-1013). Retinoic acid causes a differential gene expression by activating the genes controlling the pathways involved in retinoic acid esterification, synthesis from its precursor(s), and metabolism (Lee D, et al. Retinoid-responsive transcriptional changes in epidermal keratinocytes. J Cell Physiol 2009; 220: 427-439); simultaneously, retinoic acid down-regulates the expression of the genes encoding for lipid metabolism during keratinocyte differentiation. In contrast to retinoic acid retinol has been proven to improve significantly wound healing (Keleidari B, et al. The effect of vitamin A and vitamin C on postoperative adhesion formation: A rat model study. J Res Med Sci 2014; 19: 28-32).

Retinol is metabolically transformed into retinoic acid, especially in vivo by an enzymatically mediated two-step conversion via retinal and causes a differential gene expression within the retinoic acid nuclear receptors complex (Wang L, et al. Regulation of alpha 2(I) collagen expression in stellate cells by retinoic acid and retinoid X receptors through interactions with their cofactors. Arch Biochem Biophys 2004; 428: 92-98). In response cellular proliferation and differentiation is modulated.

A new aspect in wound healing via restoration of endothelial progenitor cell functions is the finding that leptin, administered systemically and topically improves re-epithelialization of wounds in mice (Frank S, et al. Leptin enhances wound re-epithelialization and constitutes a direct function of leptin in skin repair. J Clin Invest 2000; 106: 501-509). In addition, it has been shown that keratinocytes, located at the wound margins, express the leptin-receptor during the repair phase. Furthermore, leptin elicits a mitogenic stimulus to human keratinocyte cells in vitro. Those data are corroborated by the finding that ob/ob (leptin null), and db/db (leptin receptor null), mouse strains show severe deficiencies in cutaneous wound repair (Lee G H, et al. Abnormal splicing of the leptin receptor in diabetic mice. Nature 1996; 379: 632-635; Friedman J M, Halaas J L. Leptin and the regulation of body weight in mammals. Nature 1998; 395, 763-770).

The technique of electrospinning can be used to fabricate three-dimensional (3D) porous mats consisting of loosely connected fibers with a high surface area. This technology utilizes electrical forces to produce polymer fibers with diameters ranging from 2 nm to several micrometers (reviewed in: Agarwal S, Wendorff J H, Greiner A. Progress in the field of electrospinning for tissue engineering applications. Adv Mater 2009; 21: 3343-3351; Baji A, et al. Electrospinning of polymer nanofibers: Effects on oriented morphology, structures and tensile properties. Composites Sci Technol 2010; 70: 703-718).

Previously, the inventors described a nanoparticulate material consisting of a calcium salt of polyP that is amorphous, nano- or microsized, biodegradable and retains the morphogenetic activity of the inorganic polymer (Müller W E G, et al. A new polyphosphate calcium material with morphogenetic activity. Materials Lett 2015; 148, 163-166; Patent application GB 1420363.2 (2014), incorporated by reference. The polyP nano- or microparticles that form this material can be designated as amorphous Ca-phosphate nanoparticles [aCa-polyP-NP].

Further, the inventors described a method to include retinol into those nano- or microparticles to fabricate nano- or microspheres, consisting of retinol inclusions encapsulated within Ca-polyP shells. These nano- or microspheres are termed amorphous Ca-polyP/retinol nano- or microspheres [retinol/aCa-polyP-N/MS]. This material causes collagen type III expression at concentrations at which the single components, retinol and aCa-polyP-N/MP, are biologically inactive (GB 1502116.5). In addition, the following patent applications on polyP are regarded as being of relevance: GB1406840.7 and GB1403899.6.

Further studies that are relevant revealed that polyP comprises an antibacterial activity (Lorencová E, et al. Antibacterial effect of phosphates and polyphosphates with different chain length. J Environ Sci Health A Tox Hazard Subst Environ Eng 2012; 47: 2241-2245).

### DETAILED DESCRIPTION OF THE INVENTION

The invention is based on two general methods.

A) The method for the production of nano- or microparticles from Na-polyP and calcium salts that (i) retain after particle formation their amorphous state and (ii) display the morphogenetic activity known from Na-polyP (GB 1420363.2, the content of which is herewith incorporated by reference). The polyP material formed by the latter particles, aCa-polyP-NP, is characterized by the following properties. This material:

a) is amorphous (non-crystalline); b) has an unusual hardness (e.g. elastic modulus of 1.3 GPa [Ca-polyP2 particles with a phosphorus:calcium ration of 1:2]); c) consists of nano- or microparticles with a diameter of about 0.2 μm (Ca-polyP2 particles); d) can be prepared under mild conditions (room temperature); e) is morphogenetically active (induction of bone alkaline phosphatase activity and bone hydroxyapatite formation); and f) is biodegradable (degradation by alkaline phosphatase)

B) The method for the production of nano- or microspheres consisting of nano- or microparticles together with retinol (retinol/aCa-polyP-NS) (GB1502116.5 the content of which is herewith incorporated by reference), which show the following properties:

1. Highly homogenous in size (size ˜45 nm). This size is optimal for endocytotic cellular uptake (Zhang S, et al. Size-dependent endocytosis of nanoparticles. Adv Mater 2009; 21:419-424); in contrast to the nanoparticles, aCa-polyP-NP, which are larger (>50 μm sized) brick-like particles. 2. A synergistic action of both components, retinol and polyP. 3. A synergistic effect of both components, retinol and polyP, on the expression of collagen types I and II, and especially collagen type III. 4. A disintegration by enzymatic hydrolysis in the extracellular space and release of polyP (or its hydrolysis product, orthophosphate) and retinol. 5. An appropriate size to be taken up by a clathrin-mediated endocytosis. 6. A dual biological effect of the two active components, polyP and retinol, via the extracellular route (activation of cell membrane-bound receptors) and via the transmembrane/intracellular route (through endocytosis); and 7. No cytotoxicity.

So far, only a few wound dressings have been developed that combine both prevention of microbial infiltration and stimulating wound cell regeneration (Abrigo M, McArthur S L, Kingshott P. Electrospun nanofibers as dressings for chronic wound care: advances, challenges, and future prospects. Macromol Biosci 2014; 14: 772-792). At the present state of the art, no bioactive electrospun meshes had been developed (Abrigo M, McArthur S L, Kingshott P (2014) Electrospun nanofibers as dressings for chronic wound care: advances, challenges, and future prospects. Macromol Biosci 14:772-792).

The present inventors in a first aspect of the invention succeeded to develop meshes/mats that combine antibacterial properties with a morphogenetic activity for the cells involved in wound healing. They encapsulated retinol into nano- or microspheres, formed from polyP nano- or microparticles, and succeeded to fabricate them into electrospun fibers made of poly(D,L-lactide) (PLA). Surprisingly, those mats retain the morphogenetic activity, to increase the expression of the fatty acid binding protein 4 (FABP4) as well as of leptin and its receptor, elicited by the retinol/polyP nano- or microspheres that are not embedded into PLA.

The inventors also demonstrated that polyP, fabricated as nano- or microparticles, if administered together with retinol and packed into fibrous mats elicit a synergistic expression of genes, encoding for leptin/leptin receptor as well as for the FABP4 in MC3T3-E1 cells. Both cascades are involved in the regulation not only of the overall energy metabolism of cells but also in cell growth and differentiation control.

The invention described here relates to a method for the incorporation of the above nano- or microspheres into electrospun fibers consisting of PLA under formation of 3D mats suitable as antimicrobial and regenerative dressings for wound healing. The inventive material, consisting of 3D electrospun PLA fiber mats with incorporated amorphous retinoid/Ca-polyP nano- or microspheres can be prepared according to this invention as follows:

a) Mixing of the fiber material with an emulsifier and dissolving the mixture in an organic solvent,

b) Addition of the second synergistically acting, biologically active component,

c) Addition of amorphous calcium polyP nano- or microparticles (aCa-polyP-N/MP), and

d) Electrospinning of the mixture.

This procedure is performed at room temperature, and preferably under the avoidance of light.

As a preferred example, the preparation of 3D electrospun PLA fiber mats with incorporated amorphous retinol/Ca-polyP nano- or microspheres (retinol/aCa-polyP-N/MS) can be carried out using poly(D,L-lactide) (PLA) a fiber material, poly(ethylene glycol) as an emulsifier, isopropanol as an organic solvent, Na-polyP as a polyP salt (chain length: about 30 phosphate units), calcium chloride as a calcium salt and retinol as the second synergistically acting, biologically active component as follows.

A) Preparation of the nano- or microparticles:

i) Drop-wise addition of 2.8 g of CaCl₂ in 30 ml distilled water to 1 g of Na-polyP, dissolved in 50 ml distilled water at room temperature,

ii) Adjustment of the pH, during the procedure, to 10.0 with a NaOH aqueous solution, iii) Stirring for 12 h,

iv) Collection of the resulting aCa-polyP-NP nano- or microparticles (ratio:phosphate:Ca²⁺=2) by filtration; and

v) Drying of the nano- or microparticles at 50° C.

B) Preparation of the PLA fibers and incorporation of the nanoparticles:

i) Mixing of PLA with PEG (in a ratio 80:20 wt %) and then addition to chloroform at a final concentration of 20% (w/v),

ii) Stirring of the PLA/PEG-chloroform suspension at 40° C. for 6 h, until complete dissolution of the polymers is obtained,

iii) Addition of retinol to the PLA solution to give a final concentration of 20 wt % (with respect to PLA),

iv) Addition of the Ca-polyP nano- or microparticles, aCa-polyP-N/MP, to the PLA solution, containing retinol, at a fixed concentration of 10 wt %, and

v) Sonication of the suspension for 15 min immediately prior to electrospinning

C) Electrospinning

a) Pouring the solutions into the plastic syringe, equipped with a blunt-ended stainless steel needle (18-gauge) which is connected to a high-voltage supply,

b) Spinning of the solutions at a feed rate of 1 ml per h and at a voltage of 17 kV; during the process the distance between nozzle and the collector is kept at 150 mm,

c) Use of a metallic net is used as the collector in all cases, except for the electrospinning of the mats, and

d) Drying of the fiber mats removed from the collectors overnight.

The chain lengths of the polyP molecules can be in the range of about 3 to up to about 1000 phosphate units. Optimal results were achieved with polyP molecules with an average chain length of approximately 30 phosphate units.

“About” or “approximately” shall generally mean+/−10% of a given value.

The technology according to this invention can be used for the fabrication of a wound dressing or a component of a wound dressing. The PLA-based fiber mats, containing retinol and polyP nano- or microparticles (amorphous calcium polyphosphate nano- or microspheres; retinol/aCa-polyP-N/MS) function as bioactive meshes. Such meshes are urgently needed as dressings for chronic wounds.

A further aspect of the invention concerns the application/use of the material obtained by the methods described above for a medical treatment that is based on the advantageous induction of leptin and leptin receptor. Preferred is a treatment, wherein said medical condition is selected from selected from insufficient wound healing.

The expression of leptin and of the leptin receptor elicited by the PLA-based fiber mats, containing retinol and polyP nano- or microparticles, is a novel type of dressing with regenerative, wound-healing properties. Materials that can be incorporated into wound dressings to increase the expression of these genes are not yet known. Surprisingly the inventors found that the aCa-polyP-NP nano- or microparticles containing encapsulated retinol (retinol/aCa-polyP-NS) increase the expression of both genes in a synergistic manner.

A further aspect of the invention concerns the application of the inventive nano- or microspheres in drug delivery.

The inventive method described here further relates to innovative uses of the amorphous calcium polyphosphate material, in the form of microparticles with a size range of around 300 nm (diameter), for the (re)sealing of dentinal tubules exposed at the tooth surface and the filling of tooth defects (defects in tooth enamel, cementum and dentin). This invention is based on the unexpected finding by the inventor that these Ca-polyP microparticles are able to form a tightly bound polyP layer onto the HA surface. This finding was surprising because the calcium salt or calcium complex of polyP do not bind to these surfaces. A possible explanation might be the existence of free ionic valencies at the surface of the microparticles that are not saturated by calcium ions and can interact with surface-exposed calcium of the HA material, if a phosphate to calcium ratio of 1:1 has been used during preparation of the particles.

A further aspect of this invention concerns the finding that these calcium polyP microparticles are able to stimulate the differentiation of osteoblast precursor cells to mature osteoblasts (expressing the enzyme alkaline phosphatase which is involved in HA formation).

It was also unexpected that the Ca-polyP microparticles display such bioactivity although their diameter (300 nm) is outside the range allowing receptor-mediated endocytosis (around 50 nm).

The inventive method can be used for resealing dentinal tubules exposed to the tooth surface and coating of teeth to ameliorate tooth hypersensitivity and to prevent tooth decay (application for caries prophylaxis). The inventive method can also be used for the preparation of tooth implants that trigger the body's own tooth material (HA) formation via induction of differentiation of osteoblast precursor cells and activation of mature osteoblasts.

The polyP layer formed on the tooth surface was demonstrated to have a hardness and elastic modulus similar like natural enamel.

The preferred average size (diameter) of the Ca-polyP microparticles used in the inventive method is in the range of about 50 to about 500 nm, preferably 300 nm.

The preferred composition of the Ca-polyP microparticles used in the inventive method is a weight ratio of about 0.1 to about 10 (phosphate to calcium), preferably of 0.5 to 1, and most preferred 1 to 1.

The chain lengths of the polyP component of the Ca-polyP microparticles can be in the range 3 to up to 1000 phosphate units. Optimal results are achieved with polyP molecules with an average chain length of approximately 200 to 20, and optimally about 40 phosphate units.

The polyP material is biodegradable and displays superior morphogenetic activity, compared to the Ca-polyP salts prepared by conventional techniques.

A further aspect of the inventive method is the application/use of this method to ameliorate dental hypersensitivity or for caries prophylaxis.

Another aspect of the inventive method is the application/use of this method for preparation of tooth implants that stimulate differentiation and activation of odontoblast precursor cells and odontoblasts.

The invention will now be described further in the following preferred examples, nevertheless, without being limited thereto. For the purposes of the present invention, all references as cited herein are incorporated by reference in their entireties. In the Figures listing,

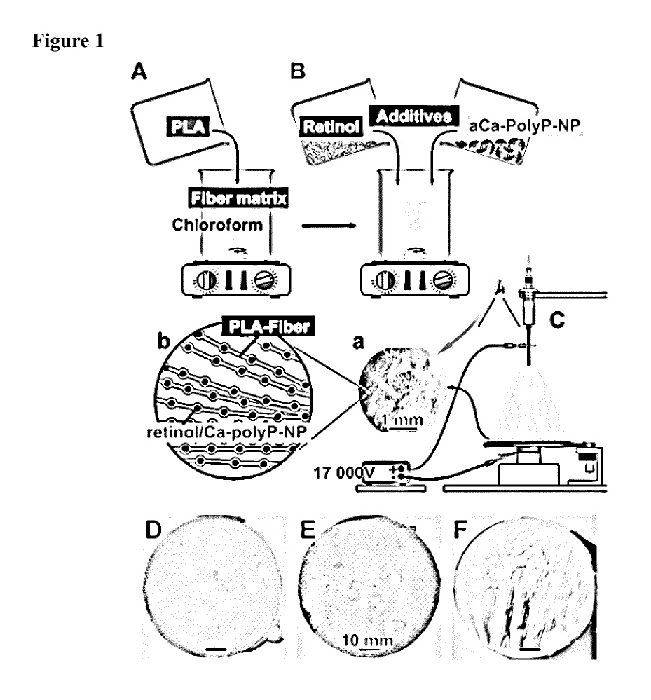

FIG. 1 shows the preparation of PLA-based electrospun mats. (A) PLA, the basic fiber matrix, is dissolved in chloroform. (B) Retinol (20 wt %) is added to this solution, followed by addition of the nano- or microparticles, aCa-polyP-N/MP (20 wt %). After a short stirring period, the suspension is sonicated immediately prior to (C) electrospinning A spun mat is shown in the insert (a), a SEM image; an enlarged schematic outline of the fibers with their integrated retinol/Ca-polyP nanoparticles (retinol/Ca-polyP-NP) is depicted in (b). Fabricated mats are shown that are formed of (D) PLA alone, (E) PLA and retinol and (F) PLA, retinol and Ca-polyP.

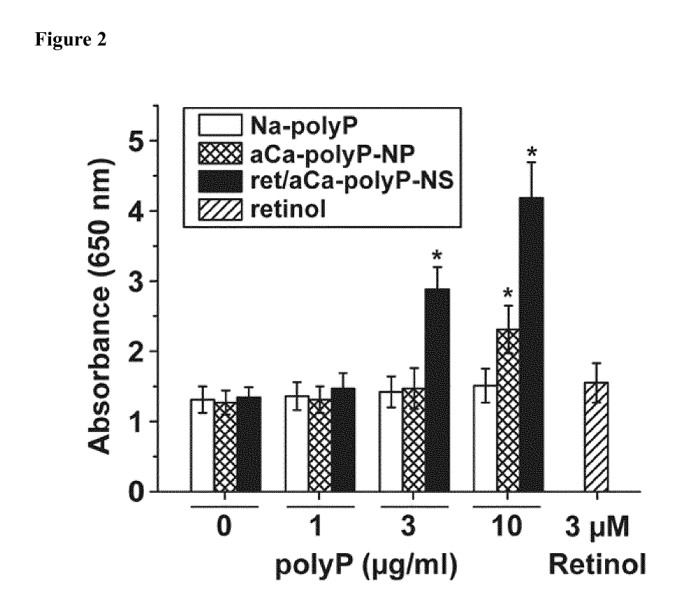

FIG. 2 shows the influence of polyP, encapsulated in nanoparticles or together with retinol in nanospheres, on cell viability (XTT colorimetric assay). The MC3T3-E1 cells were incubated with different concentrations of polyP (given in μg/ml) or retinol (μM), as indicated, for 72 h. Then the cells were assayed with XTT. The cells were exposed to different concentrations of Na-polyP, complexed with Ca²⁺, aCa-polyP-NP nanoparticles containing only polyP, retinol/aCa-polyP-NS nanospheres that contain in addition of polyP retinol, as well as with retinol. The results are expressed as means (n=10 experiments each)±standard error of the mean; *p<0.01.

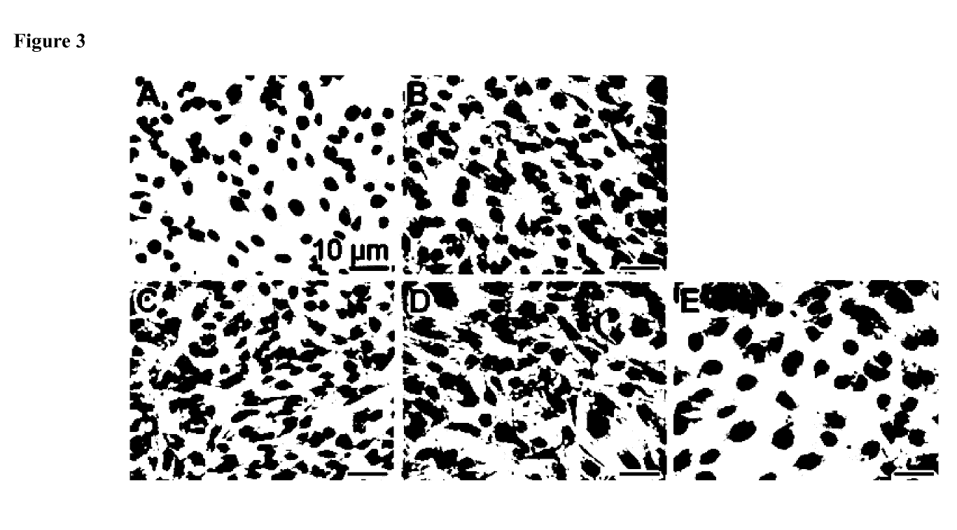

FIG. 3 shows the staining of MC3T3-E1 cells with Nile Blue A. The cells were incubated for 72 h (A) without addition of any component, or with 3 μg/ml of (B) Na-polyP, (C) aCa-polyP-NP or (D) retinol/aCa-polyP-NS, as well as with (E) 3 μM retinol. It is seen that the polyP-retinol-treated cells highlight in pink, while the controls appear in blue.

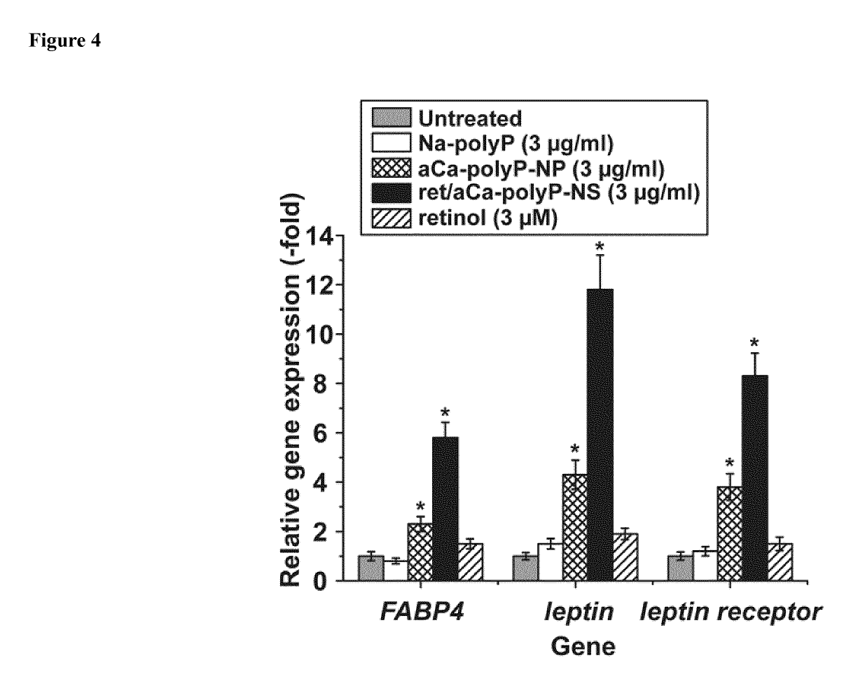

FIG. 4 shows steady-state-expression of the genes encoding the FABP4 receptor, as well as leptin or leptin receptor determined in untreated MC3T3-E1 cells (stippled bars) or in cells exposed for 3 d either to 3 μg/ml of soluble Na-polyP (open bars), aCa-polyP-NP nanoparticles (cross hatched), retinol/aCa-polyP-NS nanospheres (black bars) or to 3 μM retinol (hatched). After incubation RNA was isolated from the cultures and analyzed by RT-qPCR by using GAPDH as reference gene. The expression ratios were correlated to GAPDH in the respective culture series; the ratio obtained was set to 1. SD are shown (5 experiments/time point); *p<0.01.

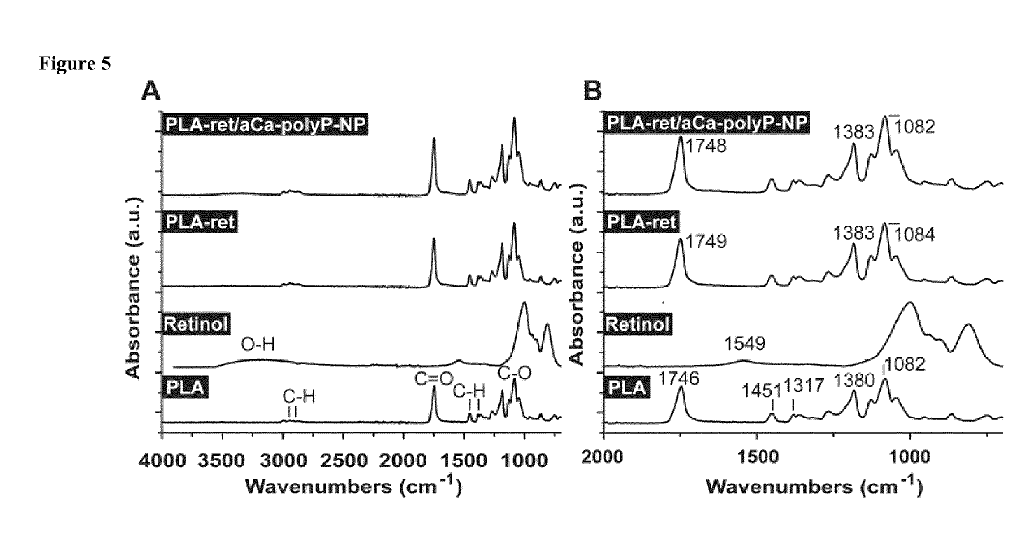

FIG. 5 shows the FTIR spectra of PLA (PLA), and retinol (Retinol) as well as of PLA, containing 20 wt % retinol (PLA-ret), and finally of PLA, composed of retinol and aCa-polyP-NP nanoparticles (PLA-ret/aCa-polyP-NP), as described under “Materials and Methods”. The spectra were recorded between (A) wavenumbers 4000 to 775 cm⁻¹ and (B) wavenumbers 2000 and 775 cm⁻¹.

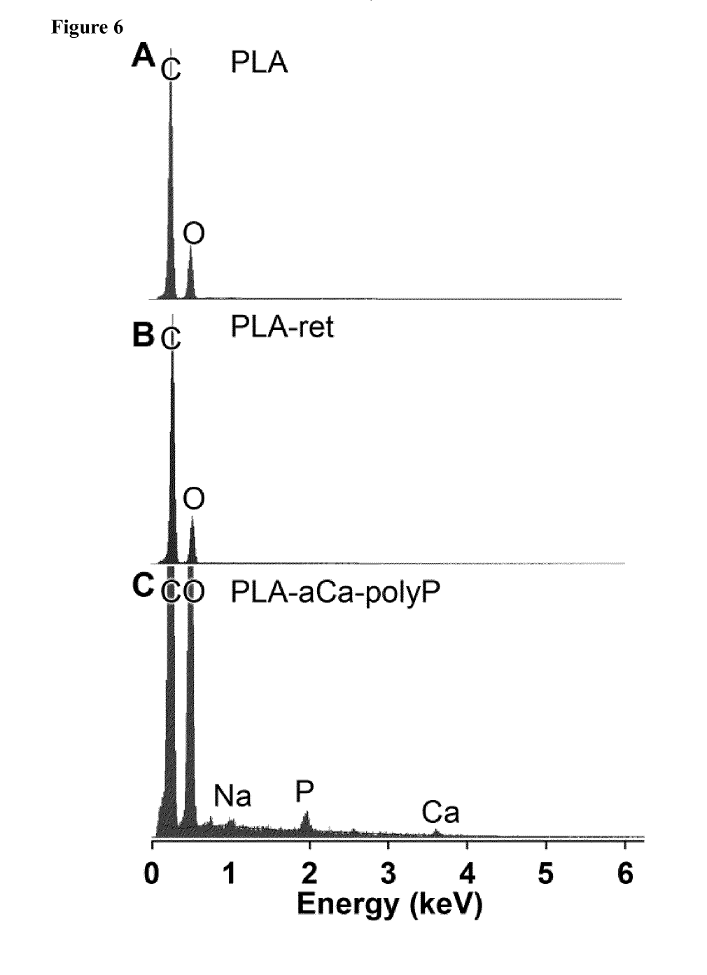

FIG. 6 shows the EDX analysis of the polymer used for electrospinning. The analysis was performed with (A) PLA polymer only, (B) PLA fibrous material that contained in addition retinol, or (C) PLA that has been supplemented with aCa-polyP-NP nanoparticles prior to the spinning process. The signals for C, O, Na, P and Ca are marked.

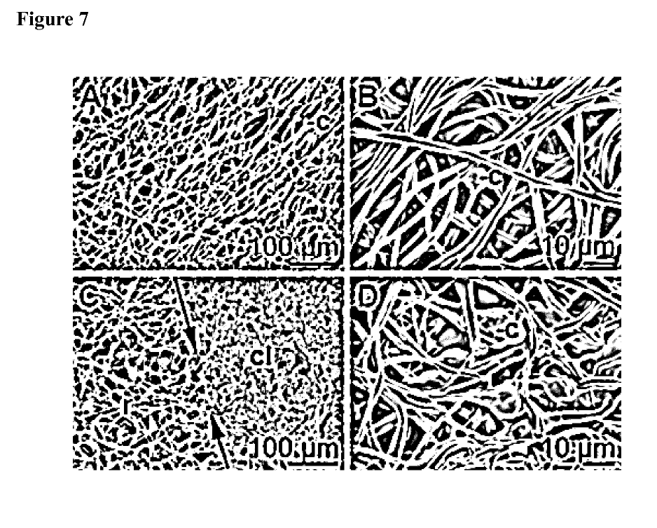

FIG. 7 shows the SEM analysis of fibrous mats after cultivation of cells. Precisely fitting mat samples were cut and submersed into the 24-well plates; 5·10³ cells/well were seeded and incubation was performed for 3 d. Then the mats were taken, fixed and dehydrated and subsequently subjected to SEM analysis. (A and B) Fibrous mats spun with PLA only; very rarely cells/cell clusters could be identified (−c). (C and D) In contrast, dense cell layers (cl) are seen that have been formed during the incubation period onto fibers formed from PLA/retinol/aCa-polyP-NS. Occasionally, within the rim region (r), the transition from the densely packed cells (in C at the right) to the less populated areas can be resolved (left). The dividing line is marked by two single headed arrows.

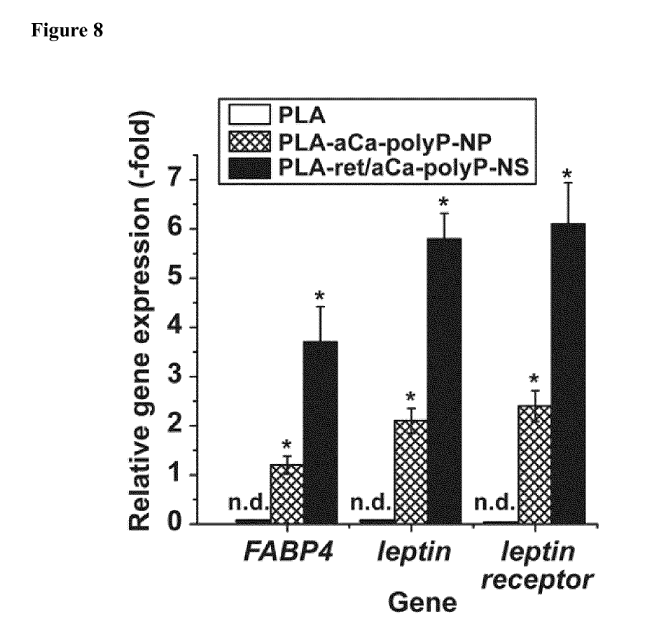

FIG. 8 shows the RT-qPCR analyses to assess the number of transcripts for the three genes FABP4, leptin, and leptin receptor. After seeding, the MC3T3-E1 cells were cultivated for 3 d onto mats fabricated from PLA only (numbers not determinable [n.d.]), onto spun fibers made of PLA and aCa-polyP-NP (cross hatched bars), or of PLA and retinol/aCa-polyP-NP (black bars). The expression values for FABP4, leptin, and leptin receptor were referred to the GAPDH expression. In turn, those numbers were correlated to the expression values of the genes under study and GAPDH, measured in cells that have been used for seeding (after detachment from the culture bottoms and a subsequently handling period for 2 h); those ratios were set to 1. SD are shown (5 experiments/time point); *p<0.01

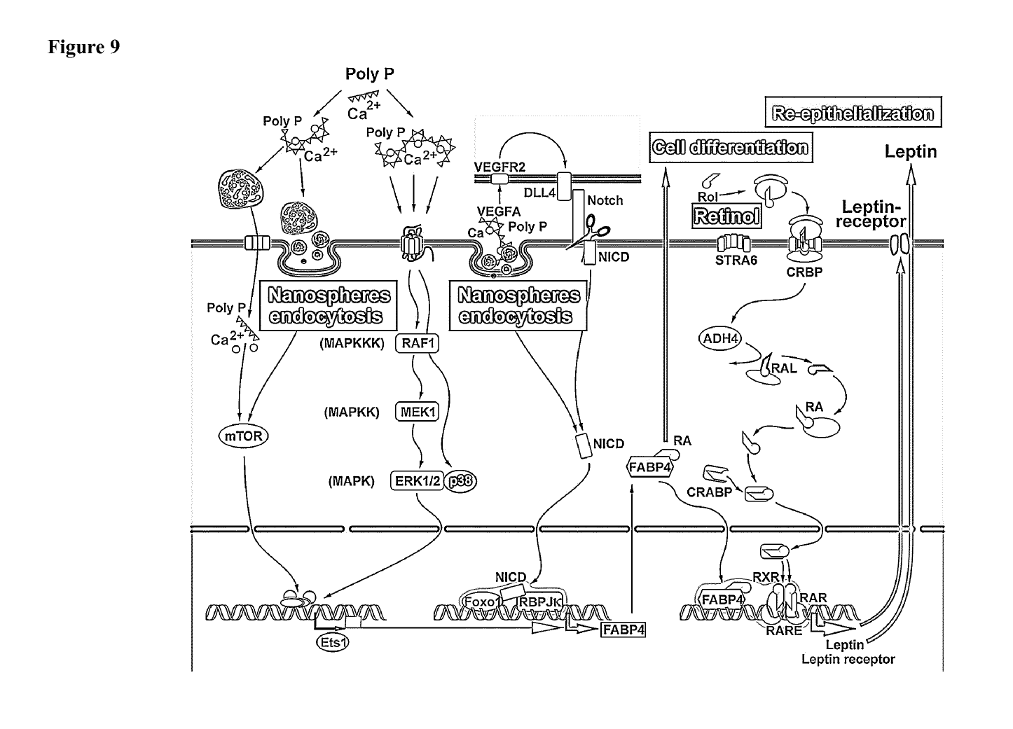

FIG. 9 shows the proposed effect of polyP and retinol on the expression of the fatty acid binding protein 4 (FABP4) and leptin/leptin receptor. It has been proposed that polyP acts via the MAPK signaling route (p38) MAPK pathway and/or the mTOR complexes 1 and 2. The FABP4 expression might include the transcription factor Etsl. This pathway acts positively on the FABP4 expression level that is correlated with the polyP/DLL4-NOTCH signaling pathway as well as with Foxol. VEGFA (vascular endothelial growth factor) induces, after binding to its receptor (VEGFAR) and activation of DLL4 (delta-like ligand-4), the paracrine activation of NOTCH signaling, resulting in FABP4 expression. Intermediately, the intracellular domain (NICD) of the NOTCH receptor translocates to the nucleus and bind to the transcription factor complex, composed of Foxol and the DNA-binding protein RBP-Jκ, through which FABP4 transcription is induced. FABP4 protein migrates into the cytoplasm and binds RA (retinoic acid). It is proposed that RA with FABP4 re-enters the nucleus and forms the RXR-RAR (retinoid X receptor-retinoic acid receptor heterodimer. In this transcription complex, at the RA response element (RARE), the genes encoding for leptin and leptin receptor are increasingly transcribed. RA is synthesized via retinol (Rol), that is taken up by the cells via STRA6 (membrane transporter) where it is bound to CRBP (cellular retinol-binding protein), undergoes oxidation by the ADH4 (alcohol dehydrogenase 4) to RAL (retinal) and finally to RA, which is finally translocated via CRABP (a retinoic acid-binding protein) to the nucleus where it binds at the RARE (retinoic acid response element) of the DNA under formation of the RXR/RAR complex. In conclusion, polyP causes an upregulation of the binding protein FABP4 which accelerates the retinol effect on the expression of leptin and leptin receptor. These two arms, FABP4 increased synthesis and RAR transcriptional promoting activity, positively affect cell differentiation and re-epithelization

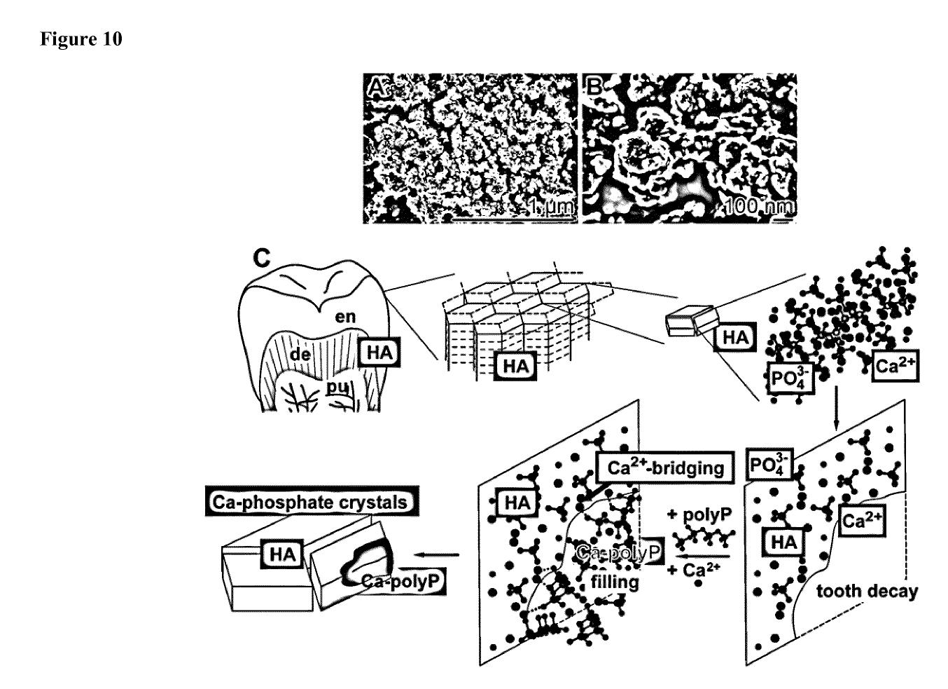

FIG. 10 shows the amorphous Ca-polyP microparticles (aCa-polyP-MP) and their proposed interaction with the Ca-phosphate surface of the teeth. (A and B) The aCa-polyP-MP; SEM analysis. (C) Proposed interaction of the microparticles with the hydroxyapatite (HA) enamel of a tooth. Enamel (en) forms the crown around the dentin (de) region and surrounds the dental pulp (pu). The minerals enamel and dentin are composed of HA plates, built mainly of PO₄³⁻ and Ca²⁺ ions. Into an existing dental cavity (caries, or tooth decay) the aCa-polyP-MP are filled. It is proposed that the Ca²⁺ ions within the microparticles form a bridging to the HA of the enamel.

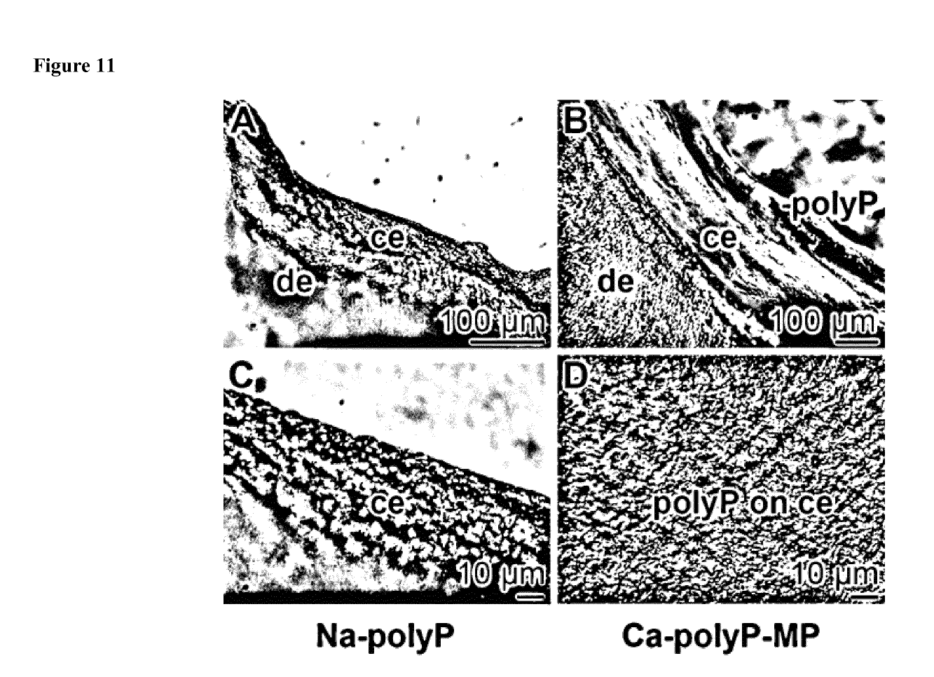

FIG. 11 shows the coating of teeth specimens from the root region with polyP. Teeth samples were incubated in 10 mg/mL of either Na-polyP [Ca²⁺] (A and C) or aCa-polyP-MP (B and D) for 2 d. Then the samples were taken, subjected to slicing and inspected by light microscopy. Images were taken either from the cut areas (A and B) or the corresponding surfaces (C and D). The different layers, cement (ce) and dentin (de) as well as the polyP layer are marked.

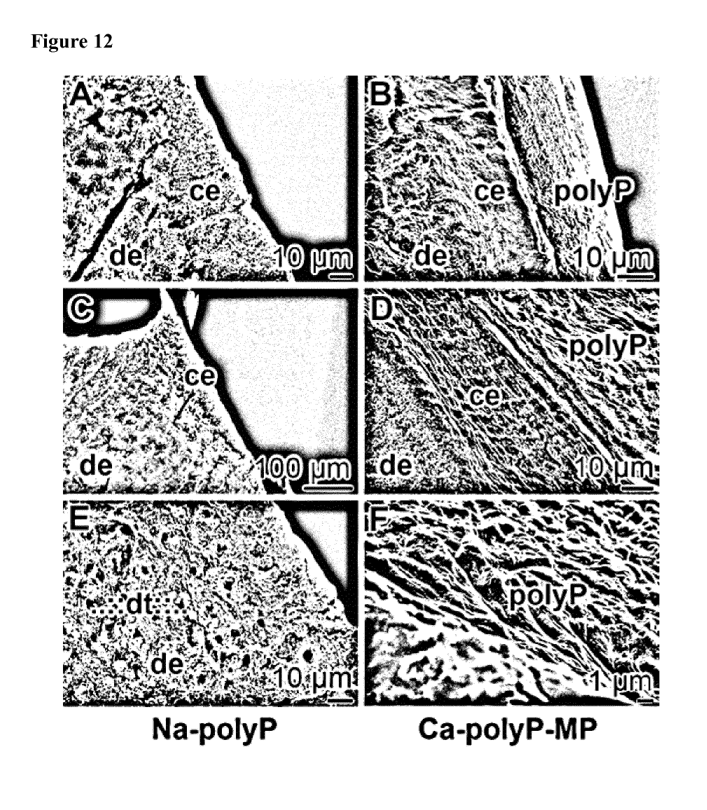

FIG. 12 shows the formation of a polyP layer onto the teeth specimens after incubation with aCa-polyP-MP; SEM. In parallel, teeth samples were submersed in Na-polyP [Ca²⁺] or aCa-polyP-MP (10 mg/mL each) for 2 d. Then the samples were, after washing, cut and then analyzed by SEM. The images A, C and E were taken from samples that had been exposed to Na-polyP [Ca²⁺], while those of B, D and F came from teeth samples incubated in aCa-polyP-MP. The cement (ce) and dentin (de) layers are seen in all samples, while only in those treated with aCa-polyP-MP the additional polyP layer (polyP) is seen. The dentinal tubules are exposed in the Na-polyP [Ca²⁺] sample (E; . . . ::dt:: . . . ), while no opening from the tubules are seen on the surface of the aCa-polyP-MP sample (F).

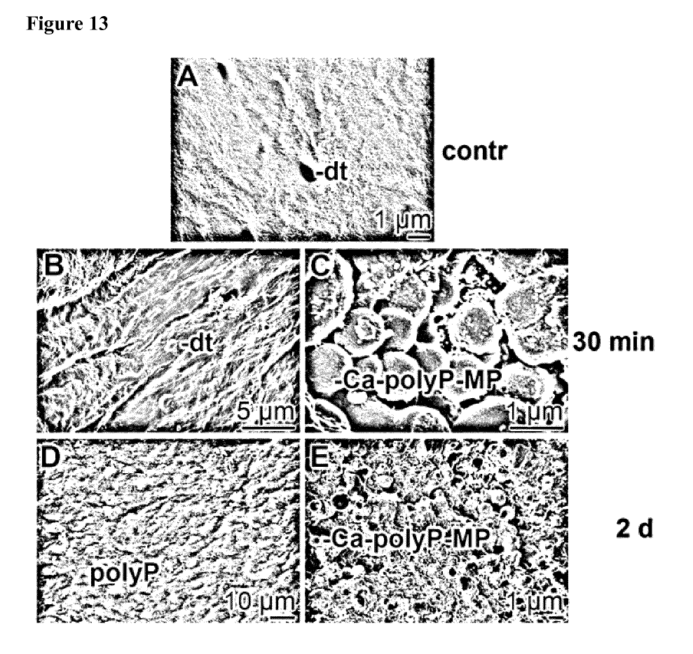

FIG. 13 shows the kinetics of coating with polyP; SEM. (A) Surface of the dentin with the opening of the dentinal tubules (dt). (B and C) Incubation of the root samples for 30 min with aCa-polyP-MP; the microparticles (Ca-polyP-MP) are accumulating in the openings of the tubules. (D and E) A longer incubation period with aCa-polyP-MP results in an expansion of the polyP deposits under formation of a layer; at higher magnification the individual microparticles can be resolved.

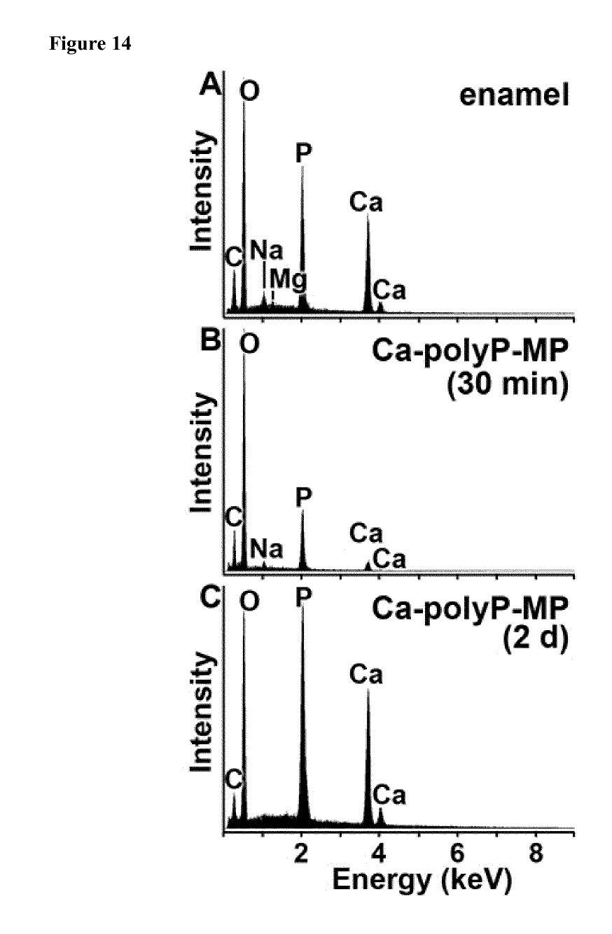

FIG. 14 shows the time course of polyP deposition onto the surface of the teeth; EDX analysis. (A) Untreated enamel. The enamel samples were treated for 30 min (B) or 2 d (C) with the aCa-polyP-MP; a strong increase of the signals for P and Ca is seen in the sample after 2 d incubation.

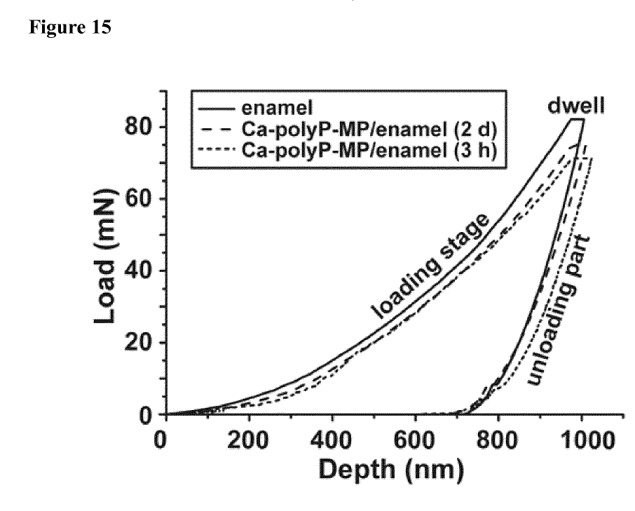

FIG. 15 shows the mechanical characteristics of the polyP coating onto enamel. Slices from human teeth were prepared and either measured directly or incubated in a saline solution supplemented with 10 mg/mL of aCa-polyP-MP. Incubation at 25° C. was performed for 3 h or 2 d. After the incubation the specimens were dried for 10 min and then measured. A typical load-penetration depth curve for a control sample (solid line) or a polyP treated sample after 2 d (broken line) or 3 h (dotted line) is shown. The indentation loads of 82 mN are given. The following load-penetration stages are marked within the curves: loading stage, dwell period at maximum load and unloading part.

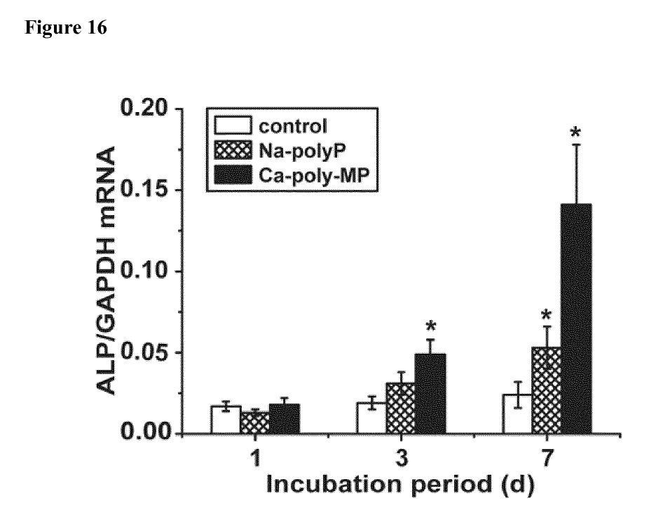

FIG. 16 shows the increase of the levels of ALP transcripts in hMSCs after exposure to polyP. The cells remained either untreated or were exposed to 30 μg/mL of Na-polyP [Ca²⁺] or aCa-polyP-MP. Samples were collected at day 1, day 3 and day 7. The cells were harvested, RNA was extracted and subjected to qRT-PCR analysis; the expression levels, correlated to the expression of the reference gene GAPDH, were determined for the untreated cells (control; open bars), as well as for the Na-polyP [Ca²⁺] (Na-polyP; cross-hatched) and the aCa-polyP-MP-treated cultures (Ca-polyP-MP; filled bars). Data are expressed as mean values±SD for four independent experiments. Differences between the groups were evaluated using the unpaired t-test. *p<0.05.

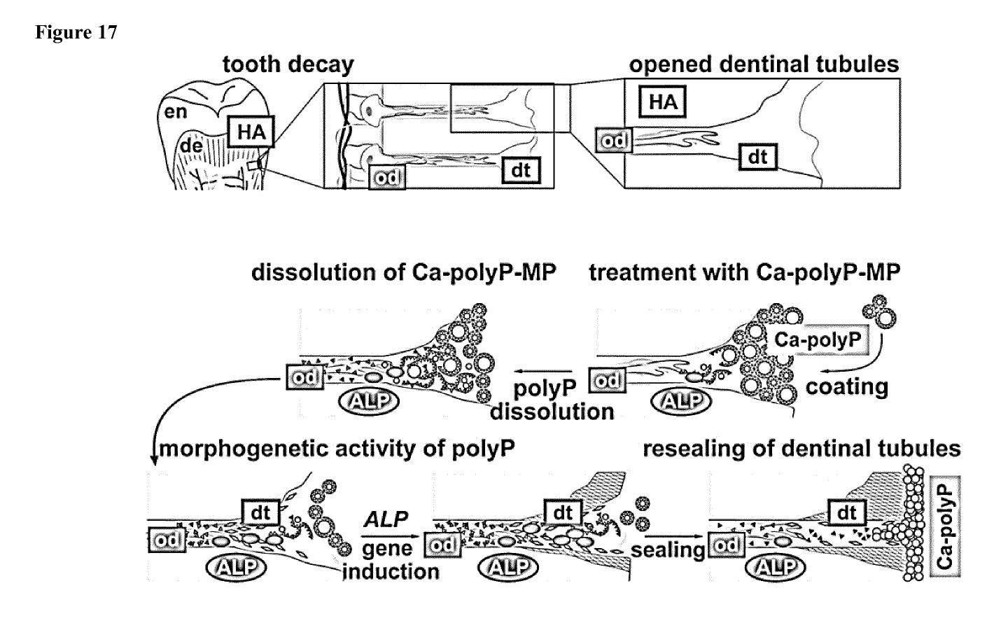

FIG. 17 represents a summary of the mode of action of the aCa-polyP-MP, which is two-fold. The microparticles attach strongly to the surface of the tooth, especially in the exposed openings of the dentinal tubules (dt). Those tubules are located in the dentin (de), which is usually covered by the enamel (en) layer. First mode of action (morphogenesis): Within the dentinal tubules the microparticles undergo hydrolysis via the enzyme ALP, which is released by the odontoblasts (od). In concert with the odontoblasts, the ALP and the hydrolysis product of Ca-polyP, the ortho-phosphate, form hydroxyapatite (HA) and by that repair the dentinal tubules. Second mode of action (resealing): After termination the microparticles (aCa-polyP-MP) form a coat onto the decayed tooth surface.

### EXAMPLES

In the following examples, only the inventive method described for polyP molecules with an average chain length of about 30 to about 40 phosphate units. Similar results can be obtained by using polyP molecules with lower and higher chain lengths, such as between 100 to 20 units.

Effect of polyP and polyP-Retinol Loaded Nanoparticles/Nanospheres on Cell Growth

In the following experiments the inventors succeeded to demonstrate that polyP and retinol, if administered together to the cells, display an interacting effect on cell growth and metabolism. Both compounds, separately added, display miscellaneous anabolic effects on cells in vitro. As examples, polyP causes an inducing effect on biomineralization/hydroxyapatite formation which is paralleled with an increased induction of the gene encoding for alkaline phosphatase (Müller W E G, et al. Inorganic polymeric phosphate/polyphosphate as an inducer of alkaline phosphatase and a modulator of intracellular Ca²⁺ level in osteoblasts (SaOS-2 cells) in vitro. Acta Biomaterialia 2011; 7; 2661-2671). Likewise, retinol contributes to the anabolic metabolic pathways in a series of processes, e.g. embryonic development or regulation of epithelial and hematopoietic cellular differentiation (Miano J M, et al. Retinoid receptor expression and all-trans retinoic acid-mediated growth inhibition in vascular smooth muscle cells. Circulation 1996; 93: 1886-1895). In the experiments described here and using MC3T3-E1 cells it was found that both particle-free Na-polyP, complexed with Ca²⁺, as well as nanoparticles, formed by amorphous Ca-polyP, have no effect on the number of cells during a 72 h incubation period below a concentration of 3 μg/ml (FIG. 2). However, if the nanoparticle preparation, aCa-polyP-NP, is added to the cells at a concentration of 10 μg/ml, a significant increase in the number of viable cells, based on the XTT reduction assay, is measured during the incubation period. Retinol, added as a single component, at a concentration of 3 μM also does not affect cell growth significantly.

If retinol is encapsulated into Ca-polyP-based nanoparticles and by that is forming the retinol/Ca-polyP nanospheres, a strong amplification of cell growth is measured (FIG. 2). Already at the concentration of 3 μg/ml the retinol/aCa-polyP-NS increase the absorbance in the XTT assay from ≈1.3 A₂₆₀ units to 2.88 units, a value which increased to 4.18 units in assays containing 10 μg/ml. The concentration of retinol in 3 μg/ml of nanospheres is approximately 0.3 μg/ml (≈1 μM), a retinol level found not to change the growth of the cells. This is a first indication reflecting that Ca-polyP together with retinol acts synergistically in the system used here.

The cells, grown as monolayers, were stained with Nile Blue A (FIG. 3) The cells were incubated with 3 μg/ml of Na-polyP, aCa-polyP-NP or retinol/aCa-polyP-NS, as well as with 3 μM retinol. In the controls (FIG. 3A) the cells are dominantly stained in blue while all cell exposed to soluble Na-polyP (FIG. 3B), to polyP nanoparticles (FIG. 3C), to nanospheres (FIG. 3D), or to retinol (FIG. 3E) highlight in light/bright pink. This result is taken as an indication that the cells incubated in the presence of polyP, as well as of retinol contain more fatty acids, chromolipids and/or phospholipids than the controls. In addition, it is apparent that the densities of the cell layers in the polyP-treated cells as well as in the retinol-treated cells are higher, compared to the controls.

Effect of polyP and Retinol on Gene Expression (FABP4, Leptin and Leptin Receptor)

The expression levels of the fatty acid binding protein 4 (FABP4) as well as of leptin and the corresponding leptin receptor were assessed by RT-qPCR, using the house-keeping gene GAPDH as a reference. The MC3T3-E1 cells remained either untreated or were exposed to 3 μg/ml of soluble Na-polyP, aCa-polyP nanoparticles or retinol/aCa-polyP nanospheres. In this series also the incubation with free retinol (3 μM) was included. The incubation period of the cells was set to 3 d. Then, the RNA was extracted and the expression levels were determined and correlated to the one of GAPDH; this ratio was set to 1.

The expression level of all three genes, FABP4, leptin, and leptin receptor, did not change significantly, with respect to the untreated control, if the cells were incubated with soluble Na-polyP, complexed to Ca²⁺ (FIG. 4). However, addition of the polyP nanoparticles, aCa-polyP-NP, caused a significant upregulation of the steady-state-expression of both the FABP4 (2.3-fold), the leptin (4.2-fold) and the leptin receptor gene (3.8-fold). Even more pronounced is the increase if the nanospheres, retinol/aCa-polyP-NS, are added to the cells; in those assays the amount of transcripts increases for FABP4 (5.8-fold), the leptin (11.6-fold) and the leptin receptor gene (8.3-fold). If the cells are exposed to retinol alone only a non-significant increase is measured (FIG. 4).

### Fabrication of the Electrospun Mats

The effect of both retinol and amorphous Ca-polyP nanoparticles, if embedded into the electrospun fibrous mats, on the expression of FABP4, leptin and leptin receptor was determined. As outlined under “Methods” the components retinol and Ca-polyP nanoparticles had been incorporated into the PLA-based fiber mesh (FIG. 1). PLA was mixed with PEG in a ratio of 80:20 wt. % and then dissolved in chloroform as outlined under “Methods”. Subsequently retinol was added at 20 wt % (with respect to PLA) to the PLA solution. In addition, the aCa-polyP-NP nanoparticles were added as well, reaching a final concentration of 10 wt %. The suspension formed was sonicated and used for the electrospinning procedure.

Mats with diameters of up to 20 cm were fabricated, woven from ≈2 μm fibers of an average mesh size of 10-20 μm. The color of the mats became yellowish if the fibers contained retinol; hence they had to be protected from light. While the pure PLA mats appeared in white (FIG. 1D), the mats spun from both PLA/retinol solution (FIG. 1E) and from PLA/retinol/aCa-polyP-NP suspension appear in yellow (FIG. 1F).

### Characterization of the Mats

FTIR analyses were conducted to assess structural changes and/or molecular chain interactions within the PLA:nanospheres fiber mats. The following samples were included: PLA basic matrix, retinol, PLA containing 20% retinol as well as PLA containing 20% retinol and 20% Ca-polyP nanoparticles. As shown in FIG. 5, all 5 samples tested caused a pronounced peak for the PLA polyester with the strong carbonyl band (C═O) at 1746/1749 cm⁻¹, a C—H bending vibrations of CH₃ at 2982 and 2930 as well as at 1445 and 1380 cm⁻¹, and the asymmetric C—O stretching vibrations at 1300-1000 cm⁻¹. In contrast to the pure PLA samples, the absorption band of C═O of the carbonyl groups in PLA shifts from 1746 cm⁻¹ to 1749 cm⁻¹ or 1748 cm⁻¹; this absorption intensity shifts also after addition of retinol and Ca-polyP (FIG. 5). Moreover, a similar shift occurred for the stretching vibrations within the wavenumber range between 1300 and 1000 cm⁻¹.

The FTIR spectrum for retinol shows the characteristic retinol band at 1549 cm⁻¹ (hydroxyl group of retinol), a signal that disappeared in the spectrum for PLA, containing retinol (FIG. 5). In addition, in retinol a wide range of signals between wavenumbers 1100 and 800 cm⁻¹. These findings indicate that PLA into which the components polyP and retinol had been encapsulated show weak interactions to the PLA matrix.

The EDX analyses of the PLA fiber material (FIG. 6A) as well as the PLA/retinol spinning material (FIG. 6B) show only the expected signals for C and O. In contrast, if the PLA—aCa-polyP-NP polymer is analyzed then the signals for Ca, P and Na appear in addition to C and O (FIG. 6C). The Na peaks reflect the residual Na⁺ that originate form the original Na-polyP material, used for the conversion to the insoluble Ca-polyP

Functional Studies of the polyP-Containing Fiber Mats

The MC3T3-E1 cells were seeded onto the circularly sliced samples of the spun mats and incubated for 3 d. In the first series of experiments the mats were taken after incubation fixed, dehydrated and then subjected to SEM analysis. The results show that almost no cells could be visualized onto those fibrous mats that had been spun with PLA alone (FIGS. 7A and B). In contrast, if the cells had been cultivated onto PLA, supplemented with aCa-polyP-NP or retinol/aCa-polyP-NP densely packed cell layer are observed (FIGS. 7C and D).

To support quantitatively the functional properties of the different mats for the cellular activity the steady-state-expression levels for the three genes, studied here FABP4, leptin, and leptin receptor, were determined by RT-qPCR analyses. The expression values have been correlated to the expression of the reference gene GAPDH; those values are subsequently proportioned to the ratio of those genes to GAPDH measured in the cells that have been used for seeding (immediately after detachment and a handling period of 2 h). It is seen that no reliable expression values of the genes in cells cultivated onto purely PLA mats can be given, due to the presence of only a few cells that survived onto those fibers (FIG. 8). In contrast, both the steady-state-expression levels for FABP4, leptin and leptin receptor undergo significant elevation onto PLA-aCa-polyP-NP fiber mats with 1.2-fold, 2.3-fold and 2.6-fold, respectively. A likewise strong induction is seen for those genes, if grown for 3 d onto PLA-retinol/aCa-polyP-NS fibers, with 3.7-fold, 5.8-fold and 6.1-fold, respectively (FIG. 8).

Based on these experiments we conclude that those PLA fibers, if supplemented with either aCa-polyP-NP or retinol/aCa-polyP-NP, elicit morphogenetic activity as monitored for the expression of the genes encoding for FABP4, leptin and leptin receptor.

Incubation of Teeth with Na-polyP Versus aCa-polyP-MP: Light Microscopy

Human teeth specimens were submersed into a solution/suspension (10 mg/mL) of Na-polyP [complexed with Ca²⁺] or aCa-polyP-MP. After an incubation period for 2 d the samples were taken, sliced inwards through the cement-dentin zones and inspected by light microscopy (FIG. 11). In the specimens, treated with Na-polyP [Ca²⁺] no additional layer was observed on the surface of the cement (FIG. 11A). No difference to sections through control, non-treated samples could be seen (images not shown). In contrast, if from a similar region a section, treated with aCa-polyP-MP, was examined a distinct 50-μm thick additional layer (polyP layer) was observed on top of the cement (FIG. 11B). Surface inspection of cement layer from the Na-polyP [Ca²⁺] treated samples shows the coarse texture (FIG. 11C), while the surface roughness of the polyP (aCa-polyP-MP)-treated teeth is much finer (FIG. 11D).

Incubation of Teeth with Na-polyP Versus aCa-polyP-MP: SEM Analysis

In parallel, the samples were examined by SEM (FIG. 12); they were treated for 2 d with the respective polyP sample. Again those specimens that were treated with 10 mg/mL of Na-polyP [Ca²⁺] did not show any additional visible layer on top of the cement (FIGS. 12A and C). In regions with only a thin cement layer, when viewed in the transversal profile, the dentinal tubules are exposed (FIG. 12E). However, if the samples treated with 10 mg/mL of aCa-polyP-MP were inspected they were covered by a ≈50 μm thick additional polyP layer (FIGS. 12B and D). No differences between the control samples, not treated with polyP, or with Na-polyP [Ca²⁺] could be detected (not shown).

The coating of the teeth with polyP during incubation with aCa-polyP-MP is dependent on the incubation period (FIG. 13). In the absence of any polyP preparation (FIG. 13A) or after incubation with Na-polyP [Ca²⁺] some dentinal tubules are seen on the surface of the dentin region (not shown) at the root part. If those teeth samples are exposed to aCa-polyP-MP for 30 min already all dentinal tubules are filled with the polymer (FIG. 13B); at a higher magnification the microparticles within the openings of those tubules can be visualized (FIG. 13C). If the incubation time is prolonged to 2 d an (almost) homogenous polyP coating can be resolved by SEM at low magnification (FIG. 13D); at a higher resolution the microparticles are visible (FIG. 13E).

EDX Analysis of the polyP Deposits

The technique of EDX spectroscopy was employed to characterize the polyP deposition onto the enamel surface of the root part of the teeth. Analyzing the element distribution of the surface of the untreated enamel shows the characteristic signals for O, P and Ca, especially representing the mineral part of the teeth, while the significant C signal reflects the organic constituents of the teeth. In addition, low amounts of Na and Mg are seen (FIG. 14A). During the short incubation period with 10 mg/mL of aCa-polyP-MP for 30 min only low signals for P and Ca are measured (FIG. 14B); however, after the 2 d incubation period the P and Ca signals substantially increased, reflecting the deposition of polyP from the microparticles (FIG. 14C).

Mechanical Properties of the polyP Coating

Hardness measurements were performed with a triangular Berkovich diamond indenter. Per given value, 30 single measurements were performed onto the (polyP) enamel. A maximum load of 82 mN was applied resulting in a displacement of 1000 nm in maximum. On average the maximum penetration depth of the indents was 250±21 nm. Within one group all load-displacement curves showed a similar shape as the one given in FIG. 15. The typical loading, hold and unloading periods in a typical test cycle of a single indent are shown for each of the three series of experiments. The contact stiffness at maximum load was calculated by fitting a power-law function under the unloading segment. In turn, the slope of the obtained function at maximum load was used to calculate the contact depth of an indent. With this parameter the Martens hardness and the reduced elastic modulus were calculated, applying the algorithms described by Oliver and Pharr (Oliver W C and Pharr G M (1992) An improved technique for determining hardness and elastic modulus using load and displacement sensing indentation experiments. J Mater Res 7:1564-1583). For the untreated enamel a Martens hardness of 4.33±0.69 GPa and a reduced elastic modulus of 101.61±8.52 GPa were calculated (average of 30 measurements). Only slightly lower were the respective values for polyP coated enamel samples; after a 3 h incubation [2 d incubation] period a Martens hardness of 3.85±0.64 [4.05±0.59] GPa and a reduced elastic modulus of 94.72±8.54 [85.62±5.33] GPa were calculated.

Functional Analysis of the aCa-polyP-MP: ALP Expression in hMSC Cells

The stromal cells, hMSC, differentiating towards odontoblasts in the presence of conditioned medium were cultivated either in the absence or presence of polyP. As polyP samples both Na-polyP [Ca²⁺] and aCa-polyP-MP were used at a concentration of 30 μg/mL. In separate assays cells were harvested after 1, 3 or 7 d of incubation for determination of the expression level of the ALP gene. The results show that in the absence of polyP the steady-state-expression level of ALP remains almost unchanged during the 7-d incubation period with a ratio to the expression of the reference gene GAPDH of approximately 0.02 (FIG. 16). In contrast, if Na-polyP [Ca²⁺] is added to the cultures a significant increase of the expression level to 0.53±0.01 is measured. A stronger effect is seen if the cells were exposed to aCa-polyP-MP. Already after a 3 d incubation period, a significant 2.6-fold increase of the level of ALP transcripts was determined, a value which further increased to 7-fold during the total 7 d incubation period.

The New aCa-polyP-MP-Based Tooth Resealing Biomaterial

The amorphous Ca-polyP microparticles (aCa-polyP-MP) strongly attach to the surface of the teeth and—undergo in the dentinal tubules hydrolysis to ortho-phosphate via the enzyme alkaline phosphatase (ALP). In turn, the products elicit morphogenetic activity during which the gene encoding for the ALP becomes induced; this process contributes to the repair of the hydroxyapatite in the decayed dentinal tubules. Finally the dentinal tubules are resealed by a layer of Ca-polyP (FIG. 17).

### Methods

### Materials

Na-polyphosphate (Na-polyP) with an average chain length of 30 to 40 phosphate units can be obtained, for example, from Merck Millipore (#106529; Darmstadt; Germany) or from Chemische Fabrik Budenheim (Budenheim; Germany), all-trans retinol, for example, from Sigma (#95144; ≧97.5%, Mᵣ 286.45; Taufkirchen; Germany).

### Preparation of Ca-polyP Nano- and Microparticles and Ca-polyP/Retinol Nanospheres

The amorphous Ca-phosphate nano- or microparticles, aCa-polyP-NP or aCa-polyP-MP, are prepared, following a described procedure (Müller W E G, et al. A new polyphosphate calcium material with morphogenetic activity. Materials Lett 2015; 148: 163-166; GB 1420363.2) with slight modifications. aCa-polyP-NP: In brief, 2.8 g of CaCl₂ in 30 ml distilled water is added drop-wise to 1 g of Na-polyP, dissolved in 50 ml distilled water at room temperature. During the procedure the pH is adjusted to 10.0 with a NaOH aqueous solution. After stirring for 12 h the nanoparticles are collected by filtration through Nalgene Filter Units (pore size 0.45 μm; Cole-Parmer). The resulting aCa-polyP-NP (ratio:phosphate:Ca²⁺=2) are dried at 50° C. aCa-polyP-MP: In brief, 10 g of Na-polyP are dissolved in distilled water and added to 14.2 g of CaCl₂.2H₂O at room temperature. During the preparation the pH is adjusted to 10.0. After stirring (4 h), the particles are collected, washed with ethanol and dried at 60° C.

The amorphous retinol/Ca-polyP nanospheres, retinol/aCa-polyP-NS, are prepared under avoidance of light. A retinol solution (100 mg/50 ml absolute ethanol), containing 2.8 g of CaCl₂, is added drop-wise to a Na-polyP solution (1 g in 100 ml water). To avoid phase separation 2 g of poly(ethylene glycol) (PEG) (P5413; Sigma-Aldrich; average mol wt 8,000) are added to the Na-polyP solution. After stirring for 6 h, the particles formed are collected by filtration. The nanospheres are found to contain 100 mg/g retinol (≈1 μM), applying the SbCl₃-based spectroscopic technique (Subramanyam G B, Parrish D B. Colorimetric reagents for determining vitamin A in feeds and foods. J Assoc Off Anal Chem 1976; 59: 1125-1130). In contrast to the nanoparticles, formed without retinol (aCa-polyP-NP), the nanospheres are colored in light yellow.

### Chemical Characterization by FTIR

The polymer characteristics of polyP within the nano- and microparticles can be verified, for example, by application of Fourier transform infrared spectroscopy; X-ray diffraction analysis can be used prove that the material is amorphous. The average size of the microparticles is 300 nm and they vary between the size range of 100 to 600 nm (FIGS. 10A and B). Fourier transformed infrared (FTIR) spectroscopy in the attenuated total reflectance (ATR) mode can be applied, using, for example, the Varian 660-IR spectrometer with Golden Gate ATR auxiliary (Agilent). Spectra between the wavenumbers 4000 and 600 cm-1 are recorded.

### Fabrication of Poly(Lactic Acid) Fiber Mats

Poly(lactic acid)-based nanofibers are prepared as described (Müller W E G, et al. Biosilica-loaded poly(ε-caprolactone) nanofibers mats provide a morphogenetically active surface scaffold for the growth and mineralization of the osteoclast-related SaOS-2 cells. Biotechnol J 2014; 9: 1312-1321); a schematic outline is given in FIG. 1. Poly(D,L-lactide) (PLA) is obtained from Sigma (mol wt 75,000-120,000; P1691). PLA is mixed with PEG (in a ratio 80:20 wt %) and then added to chloroform at a final concentration of 20% (w/v). The PLA/PEG-chloroform suspension is stirred at 40° C. for 6 h, until complete dissolution of the polymers is obtained. Retinol is added to the PLA solution to give a final concentration of 20 wt % (with respect to PLA). Where indicated, Ca-polyP nanoparticles, aCa-polyP-NP, prepared according to Müller et al. (Müller W E G, et al. A new polyphosphate calcium material with morphogenetic activity. Materials Lett 2015; 148: 163-166), are added to the PLA solution, containing already retinol, at a fixed concentration of 10 wt %. The suspension is sonicated for 15 min immediately prior to electrospinning to assure a suitable dispersion of the nanospheres in the PLA solution.

Electrospinning is performed with the electrospinning unit (Spraybase; Profector Life Sciences, Dublin; Ireland) with some modifications, adapted to the PLA-based spinning material. The solutions are poured into the plastic syringe, equipped with a blunt-ended stainless steel needle (18-gauge) which is connected to a high-voltage supply. The solutions are spun at a feed rate of 1 ml per h and at a voltage of 17 kV; during the process the distance between nozzle and the collector is kept at 150 mm. A metallic net is used as the collector in all cases, except for the electrospinning of the mats. The fiber mats are removed from the collectors and dried overnight. During the complete procedures light exposure is avoided as so far as it is possible.

### Cultivation of MC3T3-E1 Cells

The mouse calvaria cells MC3T3-E1 cells (ATCC-CRL-2593; #99072810; Sigma) are cultivated in a-MEM (Gibco—Invitrogen, Darmstadt; Germany) containing 20% fetal calf serum (FCS; Gibco). In addition, the medium is supplemented with 2 mM L-glutamine, 1 mM Na-pyruvate and 50 μg/ml of gentamycin. The cells are incubated in 25 cm² flasks or in 24-well plates in an incubator at 37° C. and 5% CO₂. After reaching 80% confluency, the cells are detached using trypsin/EDTA and then subcultured at a density of 5·10³ cells/ml. The cells are seeded at a density of 5·10³ cells/well. Medium/serum change is every 3 d.

As indicated, the following polyP preparations are added to the cells; (i) “Na-polyP”, stoichiometrically complexed with Ca²⁺ (molar ratio of 1:2 [phosphate monomer:Ca²⁺]; Müller W E G, et al. Inorganic polymeric phosphate/polyphosphate as an inducer of alkaline phosphatase and a modulator of intracellular Ca²⁺ level in osteoblasts (SaOS-2 cells) in vitro. Acta Biomaterialia 2011; 7:2661-2671), (ii) “aCa-polyP-NP” nanoparticles, (iii) “retinol/aCa-polyP-NS” nanospheres or (iv) “retinol”. Retinol is dissolved in ethanol (1 mg/ml) and then diluted in DMSO (dimethyl sulfoxide). Incubation is performed for 3 d.

In the indicated experiments, circular fiber mat samples (diameter of 15 mm) are inserted into each 24-well of the plates and seeded with MC3T3-E1 cells at a density of 5·10³ cells/well. Then the assays are incubated for 3 d and subjected to microscopic as well as polymerase chain reaction analyses.

### Cultivation of the Human Multipotent Stromal Cells

The human multipotent stromal cells (hMSC) differentiate into odontoblasts in the presence of conditioned medium from developing tooth germ cells; the conditioned medium is prepared as described (Huo N, et al. (2010) Differentiation of dermal multipotent cells into odontogenic lineage induced by embryonic and neonatal tooth germ cell-conditioned medium. Stem Cells Dev 19:93-104). The description of the cultivation procedure for the hMSCs has been published (Wang X H, et al. (2014) The marine sponge-derived inorganic polymers, biosilica and polyphosphate, as morphogenetically active matrices/scaffolds for differentiation of human multipotent stromal cells: Potential application in 3D printing and distraction osteogenesis. Marine Drugs 12:1131-1147). The human cells can be obtained, after approval from the responsible ethics committee, from bone marrow aspirations after informed consent of the donors. Incubation was performed in a humidified incubator at 37° C. and 5% CO₂. The sixth passage is used for the studies. The cells are incubated in α-MEM (Biochrom), supplemented with 20% fetal calf serum (FCS; Gibco Invitrogen) as well as with 100 units/mL penicillin and 100 mg/mL streptomycin. In addition, 5% of conditioned medium is added to the assays.

After the third passage in the presence of the conditioned medium the cells are continued to culture in 48-well plates (Cat. no. 677102; Greiner) either in the absence of polyP or the presence of 30 μg/mL either of Na-polyP [Ca²⁺] or of aCa-polyP-MP. Then the cells are harvested for qRT-PCR analysis.

### Cell Viability Assay

Cell proliferation/cell viability can be determined by the colorimetric XTT method ([Na-3′[1-(phenylamino-carbonyl)-3,4-tetrazolium]-bis(4-methoxy-6-nitro) benzene sulfonic acid] using, for example, the “Cell Proliferation Kit II” (Roche). The absorbance is determined at 650 nm and subtracted from the background values (500 nm). In the experiments described in Examples, the viable cells have been determined after 72 h.

### Staining of the Cells

The MC3T3-E1 cells are stained with Nile Blue A (Basic Blue 12, Nile blue sulfate; Sigma N0766) as described (Nakanishi T, Kato S. Impact of diabetes mellitus on myocardial lipid deposition: an autopsy study. Pathol Res Pract 2014; 210: 1018-1025). This staining reagent highlights in histological samples neutral lipids (triglycerides, cholesteryl esters, steroids) in pink, while acids (fatty acids, chromolipids, phospholipids) are stained in blue.

### Reverse Transcription-Quantitative Real-Time PCR Analyses

The gene expression levels can be determined by applying the technique of reverse transcription-quantitative real-time polymerase chain reaction (RT-qPCR). The cells are incubated in medium/serum for 3 d in the absence or presence of 3 μg/ml of polyP (in the soluble form or in nanoparticles/nanospheres) or of 3 μM retinol as indicated with the respective experiment described under Examples. Then the cells are harvested, their RNA is isolated and subjected to RT-qPCR. In the experiments described under Examples, the following primer pairs, matching with the respective mouse genes, are used: Fatty acid binding protein 4 (Mus musculus; accession number NM 024406) Fwd: 5′-CGATGAAATCACCGCAGACGAC-3′ [nt₂₇₈ to nt₂₉₉] (SEQ ID NO: 1) and Rev: 5′-ACCACCAGCTTGTCACCATCTC-3′ [nt₄₁₂ to nt₃₉₂] (SEQ ID NO: 2); product size 135 bp; leptin (M. musculus; NM 008493) Fwd: 5′-GAAGAGACCGGGAAAGAGTGACAG-3′ [nt₂₈₈₈ to nt₂₉₁₁] (SEQ ID NO: 3) and Rev: 5′-TGACCAAGGTGGCATAGCACAG-3′ [nt₃₀₄₀ to nt₃₀₁₉] (SEQ ID NO: 4); size 153 bp; and leptin receptor, transcript variant 2 (M. musculus; NM_010704) Fwd: 5′-GTGTGAGGAGGTACGTGGTGAAG-3′ [nt₂₅₇₀ to nt₂₅₉₂] (SEQ ID NO: 5) and Rev: 5′-CCGAGGGAATTGACAGCCAGAAC-3′ [nt₂₇₀₈ to nt₂₆₈₆] (SEQ ID NO: 6); size 139 bp. The GAPDH [glyceraldehyde 3-phosphate dehydrogenase (Mus GAPDH; NM_008084) is used as reference gene Fwd: 5 ‘-TCACGGCAAATTCAACGGCAC-3’ [nt₂₀₀ to nt₂₂₀] (SEQ ID NO: 7) and Rev: 5′-AGACTCCACGACATACTCAGCAC-3′ [nt₃₃₈ to nt₃₁₆; size 139 bp] (SEQ ID NO: 8).

To quantify the expression level of the gene encoding the ALP in the hMSC, the cells were incubated for 1, 3 and 7 d in the presence of 30 μg/mL either of Na-polyP [Ca²⁺] or of aCa-polyP-MP they are harvested, RNA is isolated and qRT-PCR is performed. The primer pairs, matching with the human ALP gene (accession number NM_000478.4) Fwd: 5′-TGCAGTACGAGCTGAACAGGAACA-3′ (SEQ ID NO. 9) [nt₁₁₄₁ to nt₁₁₆₄] and Rev: 5′-TCCACCAAATGTGAAGACGTGGGA-3′ (SEQ ID NO. 10) [nt₁₄₁₈ to nt₁₃₉₅; PCR product length 278 bp] and with the reference gene GAPDH (glyceraldehyde 3-phosphate dehydrogenase; NM_002046.3) using the primer pair Fwd: 5′-CCGTCTAGAAAAACCTGCC-3′ (SEQ ID NO. 11) [nt₈₄₅ to nt₈₆₃] and Rev: 5′-GCCAAATTCGTTGTCATACC-3′ (SEQ ID NO. 12) [nt₁₀₅₉ to nt₁₀₇₈; 215 bp], can be used.

Amplification can be performed, for example, in an iCycler (Bio-Rad) applying the respective iCycler software. After determination of the Cₜ values the expression of the respective transcripts is calculated. The expression levels of the respective genes are determined and the calculated values are correlated to the expression values for genes in untreated cells; this value was set to 1.

### In Vitro Incubation of Teeth

In order to determine the efficacy of the aCa-polyP-MP to reseal the dentin layer and to check for the potency of the microparticles to occlude the dentinal tubules human teeth are used. Prior to use the teeth are mechanically cleaned from soft tissue, treated for 5 h in 3% Na-hypochlorite to remove tissue remains and then stored at 4° C. in a 100% relative humidity chamber.

Teeth specimens are submersed in saline (0.90% [w/v] NaCl) which contained, as mentioned in the text, either 10 mg/mL of aCa-polyP-MP or Na-polyP, stoichiometrically complexed with Ca²⁺ (molar ratio of 2:1/phosphate monomer:Ca² [Müller W E G, et al. (2011) Inorganic polymeric phosphate/polyphosphate as an inducer of alkaline phosphatase and a modulator of intracellular Ca²⁺ level in osteoblasts (SaOS-2 cells) in vitro. Acta Biomater 7:2661-2671]). Incubation is performed at 25° C. Then the samples are sliced by cutting 1-2 mm thick discs inwards to the pulp as described (Wang X H, et al. (2014) Enzyme-based biosilica and biocalcite: biomaterials for the future in regenerative medicine. Trends Biotechnol 32:441-447); where indicated, the dentin or enamel regions, as well as the cement layer, are included in the measurements.

### Microscopic Analyses

Scanning electron microscopy (SEM) can be performed, for example, with a HITACHI SU 8000 (Hitachi High-Technologies Europe GmbH), equipped with a low voltage (<1 kV; analysis of near-surface organic surfaces) detector. Tooth samples, after the respective incubation, are washed 3-times in PBS (phosphate-buffered saline). After a short traversing through distilled water the specimens are dried and inspected. Where mentioned the samples have been cut. Digital light microscopy can be performed, for example, with a VHX-600 Digital Microscope (Keyence) equipped with a VH-Z100 zoom lens.

### Electron Microscopy

For the scanning electron microscopic (SEM) analyses, for example, a HITACHI SU 8000 electron microscope can be employed. For the visualization of the cells, attached to the spun mats, the samples are removed after incubation and subjected to fixation, dehydration and air drying.

### Energy Dispersive X-Ray Spectroscopy

EDX spectroscopy can be performed, for example, with an EDAX Genesis EDX System attached to a scanning electron microscope (e.g., Nova 600 Nanolab; FEI) operating at 10 kV with a collection time of 30-45 s. In the experiments described under Examples, areas of approximately 10 μm² have been analyzed by EDX.

### Mechanical-Nanoindentation Determinations

The surfaces of either the untreated or the polyP-coated teeth specimens are evaluated at 25° C. by depth-sensing indentation, using, for example, a NanoTest Vantage system (Micro Materials Ltd). A three-sided Berkovich diamond indenter is used to produce triangular-shaped indentation marks on the coating surface; the tip radius measures approximately 50-100 nm. A total of 30 single measurements is performed. The loading rate as well as the unloading rate is fixed to 0.5 mN s⁻¹. To determine the “creep-effect” a 30 s dwell period is introduced at a maximum load. In the unloading curve a second dwell period (60 s) at 10% of the maximum load is used to determine the thermal drift of the system. The Martens hardness and the reduced modulus of the specimens are calculated with the unloading data according to the Oliver and Pharr Method (Oliver W C and Pharr G M (1992) An improved technique for determining hardness and elastic modulus using load and displacement sensing indentation experiments. J Mater Res 7:1564-1583). For the calculations the software “NanoTest Platform Four V.40.08 (Micro Materials Ltd)” can be used.

### Statistical Analysis

The results can be statistically evaluated using the paired Student's t-test.

1. A plurality of solid, biocompatible and biodegradable amorphous calcium polyphosphate microparticles that
i) form a tightly bound polyphosphate layer onto a hydroxyapatite (HA) surface,
ii) have a hardness and elastic modulus close to natural enamel,
iii) are able to trigger differentiation of precursor cells into odontoblasts, and
iv) activate the expression of alkaline phosphatase in precursor odontoblasts.

2. A method for preparing a three-dimensional (3D) electrospun fiber mat incorporating nano- or microspheres comprising at least one biologically active component, wherein said method comprises the steps of:
i) providing fibrous mat material, and mixing of said fiber material with an emulsifier to form a mixture,
ii) dissolving said mixture in a solvent,
iii) adding said at least one biologically active component,
iv) adding amorphous calcium polyP nano- or microparticles (aCa-polyP-N/MP) to said mixture from iii), and
v) electrospinning said mixture to form a 3D electrospun fiber mat incorporating said nano- or microspheres.

3. The amorphous calcium polyphosphate microparticles according to claim 1, wherein said calcium polyphosphate microparticles are characterized by a weight ratio of 0.1 to 10 (phosphate to calcium).

4. The amorphous calcium polyphosphate microparticles according to claim 1, wherein the chain length of the polyphosphate is about 3 to about 1000 phosphate units.

5. The amorphous calcium polyphosphate microparticles according to claim 1, wherein the average size of the calcium polyphosphate microparticles is about 50 to about 500 nm.

6. The method according to claim 2, wherein said 3D electrospun fiber mat comprises poly(D,L-lactide) (PLA).

7. The method according to claim 2, wherein said emulsifier is poly(ethylene glycol).

8. The method according to claim 2, wherein PLA is mixed with PEG in a ratio of 80:20 wt %.

9. The method according to claim 2, wherein said organic solvent is isopropanol.

10. The method according to claim 2, wherein said biologically active component is retinol.

11. The method according to claim 10, wherein said retinol is added at 20 wt % (with respect to PLA) to the PLA solution.

12. The method according to claim 2, wherein said amorphous Ca-polyP nano- or microparticles are added up to a final concentration of 10 wt %.

13. A method for resealing dentinal tubules to ameliorate dental hypersensitivity, wherein said method comprises applying to said tubules the amorphous calcium polyphosphate microparticles according to claim 1.

14. A method for producing a tooth implant material that stimulates differentiation and activation of odontoblast precursors cells and odontoblasts, wherein said method comprises including the solid, biocompatible and biodegradable amorphous calcium polyphosphate microparticles according to claim 1 into said tooth implant material.

15. A method for producing a toothpaste that stimulates differentiation and activation of odontoblast precursors cells and odontoblasts, and/or that reseals dentinal tubules, thereby ameliorating hypersensitivity, wherein said method comprises including the solid, biocompatible and biodegradable amorphous calcium polyphosphate microparticles according to claim 1 into said toothpaste.

16. (canceled)

17. A tooth implant material produced according to claim 6 or a toothpaste produced that stimulates differentiation and activation of odontoblast precursors cells and odontoblasts, and/or that reseals dentinal tubules, thereby ameliorating hypersensitivity, wherein said method comprises including the solid, biocompatible and biodegradable amorphous calcium polyphosphate microparticles according to claim 1 into said toothpaste.

18. A method for stimulating differentiation and activation of odontoblast precursor cells and odontoblasts and/or for resealing the dentinal tubules and, by that, ameliorating hypersensitivity wherein said method comprises the use of the tooth implant material or toothpaste according to claim 17.

19. (canceled)

20. A three-dimensional (3D) electrospun fiber mat incorporating nano- or microspheres comprising at least one biologically active component, produced by a method according to claim 2.

21. A method for preparing a wound healing material, wherein said method comprises the use of the three-dimensional (3D) electrospun fiber mat according to claim 20.

22. The method, according to claim 21, wherein said material is a wound dressing or a component of a wound dressing.

23. A method for drug delivery, wherein said method comprises the use of the three-dimensional (3D) electrospun fiber mat according to claim 20.

24. A method for the treatment of a medical condition based on induction of a leptin and/or a leptin receptor gene and/or molecule, wherein said method comprises the use of a three-dimensional (3D) electrospun fiber mat according to claim 20.

25. The method, according to claim 24, wherein said medical condition is insufficient wound healing.


SEQUENCE LISTING (12 sequences)

SEQ ID NO: 1 - DNA, 22 bp, MUS MUSCULUS
cgatgaaatcaccgcagacgac

SEQ ID NO: 2 - DNA, 22 bp, MUS MUSCULUS
accaccagcttgtcaccatctc

SEQ ID NO: 3 - DNA, 24 bp, MUS MUSCULUS
gaagagaccgggaaagagtgacag

SEQ ID NO: 4 - DNA, 22 bp, MUS MUSCULUS
tgaccaaggtggcatagcacag

SEQ ID NO: 5 - DNA, 23 bp, MUS MUSCULUS
gtgtgaggaggtacgtggtgaag

SEQ ID NO: 6 - DNA, 23 bp, MUS MUSCULUS
ccgagggaattgacagccagaac

SEQ ID NO: 7 - DNA, 21 bp, MUS MUSCULUS
tcacggcaaattcaacggcac

SEQ ID NO: 8 - DNA, 23 bp, MUS MUSCULUS
agactccacgacatactcagcac

SEQ ID NO: 9 - DNA, 24 bp, HOMO SAPIENS
tgcagtacgagctgaacaggaaca

SEQ ID NO: 10 - DNA, 24 bp, HOMO SAPIENS
tccaccaaatgtgaagacgtggga

SEQ ID NO: 11 - DNA, 19 bp, HOMO SAPIENS
ccgtctagaaaaacctgcc

SEQ ID NO: 12 - DNA, 20 bp, HOMO SAPIENS
gccaaattcgttgtcatacc